SEQUENCE LISTING (30 sequences)

SEQ ID NO: 1 - DNA, 19 bp, Artificial sequence
  tobacco ringspot virus cross primer
gatccaactatgacgtggc

SEQ ID NO: 2 - DNA, 19 bp, Artificial sequence
  tobacco ringspot virus cross primer
gcccgggaatatgaaatgg

SEQ ID NO: 3 - DNA, 19 bp, Artificial sequence
  tobacco ringspot virus cross primer
aggttgacccgtcaagttt

SEQ ID NO: 4 - DNA, 19 bp, Artificial sequence
  tobacco ringspot virus cross primer
tgacgggacctccaaaatt

SEQ ID NO: 5 - DNA, 40 bp, Artificial sequence
  tobacco ringspot virus cross primer
tgacgggacctccaaaattctaattgggtgacactttcgc

SEQ ID NO: 6 - DNA, 19 bp, Artificial sequence
  tobacco ringspot virus cross primer
ctctgccagcactgctcat

SEQ ID NO: 7 - DNA, 19 bp, Artificial sequence
  tobacco ringspot virus cross primer
cttgggaatgcaaaccacc

SEQ ID NO: 8 - DNA, 19 bp, Artificial sequence
  tobacco ringspot virus cross primer
gtcccacaacaagtgggat

SEQ ID NO: 9 - DNA, 19 bp, Artificial sequence
  tobacco ringspot virus cross primer
gctttcctttcttcgcatc

SEQ ID NO: 10 - DNA, 40 bp, Artificial sequence
  tobacco ringspot virus cross primer
gctttcctttcttcgcatcgtatgtgtgctgtgacggttg

SEQ ID NO: 11 - DNA, 19 bp, Artificial sequence
  tobacco ringspot virus cross primer
ggtgtgcaaagggcattgt

SEQ ID NO: 12 - DNA, 19 bp, Artificial sequence
  tobacco ringspot virus cross primer
cttggtaggcagttcaaac

SEQ ID NO: 13 - DNA, 19 bp, Artificial sequence
  tobacco ringspot virus cross primer
aaacctgcaaaggcgttgc

SEQ ID NO: 14 - DNA, 19 bp, Artificial sequence
  tobacco ringspot virus cross primer
atcatcaaaggtgcacgct

SEQ ID NO: 15 - DNA, 40 bp, Artificial sequence
  tobacco ringspot virus cross primer
atcatcaaaggtgcacgctcatcccacttttacagtgagg

SEQ ID NO: 16 - DNA, 19 bp, Artificial sequence
  tobacco ringspot virus cross primer
gcagatgtgcatgaatggc

SEQ ID NO: 17 - DNA, 19 bp, Artificial sequence
  tobacco ringspot virus cross primer
ccatgtccacatgcatagt

SEQ ID NO: 18 - DNA, 19 bp, Artificial sequence
  tobacco ringspot virus cross primer
tgggtaactacatttgccc

SEQ ID NO: 19 - DNA, 19 bp, Artificial sequence
  tobacco ringspot virus cross primer
cttgatttgtggacgcaac

SEQ ID NO: 20 - DNA, 40 bp, Artificial sequence
  tobacco ringspot virus cross primer
cttgatttgtggacgcaactgctatggggaacttacagga

SEQ ID NO: 21 - DNA, 19 bp, Artificial sequence
  tobacco ringspot virus cross primer
tctaggaatcttgggtggt

SEQ ID NO: 22 - DNA, 19 bp, Artificial sequence
  tobacco ringspot virus cross primer
tcaaattggccatctccgt

SEQ ID NO: 23 - DNA, 19 bp, Artificial sequence
  tobacco ringspot virus cross primer
aggtgcttcgtgttcccat

SEQ ID NO: 24 - DNA, 19 bp, Artificial sequence
  tobacco ringspot virus cross primer
cataacacctgacgtaagg

SEQ ID NO: 25 - DNA, 40 bp, Artificial sequence
  tobacco ringspot virus cross primer
cataacacctgacgtaagggtcactataagcggaagtgtc

SEQ ID NO: 26 - DNA, 21 bp, Artificial sequence
  tobacco ringspot virus cross primer
aacctatgaagagggaaatgc

SEQ ID NO: 27 - DNA, 21 bp, Artificial sequence
  tobacco ringspot virus cross primer
ccaatcctggtttgaagaatc

SEQ ID NO: 28 - DNA, 20 bp, Artificial sequence
  tobacco ringspot virus cross primer
tccatgttgtccatatctta

SEQ ID NO: 29 - DNA, 21 bp, Artificial sequence
  tobacco ringspot virus cross primer
agaagaaacactcttgacact

SEQ ID NO: 30 - DNA, 20 bp, Artificial sequence
  tobacco ringspot virus cross primer
ccgcttatagtgccagacca

(19) United States
(12) Patent Application Publication
(10) Pub. No.: US 2024/0200153 A1
(43) Pub. Date: Jun. 20, 2024

(54) CROSS PRIMER ISOTHERMAL AMPLIFICATION PRIMER SET FOR DETECTING TOBACCO RINGSPOT VIRUS, KIT AND APPLICATION THEREOF

(71) Applicant: China Jiliang University, Hangzhou (CN)

(72) Inventors: Qianqian Yang, Hangzhou (CN); Xiaoping YU, Hangzhou (CN); Pengjun ZHANG, Hangzhou (CN); Dao WANG, Hangzhou (CN); Guangfu LIU, Hangzhou (CN); Peiying HAO, Hangzhou (CN); Yipeng XU, Hangzhou (CN)

(21) Appl. No.: 17/529,206

(22) Filed: Nov. 17, 2021

(30) Foreign Application Priority Data
May 21, 2021 (CN) 2021105579538

(51) Int. Cl. C12Q 1/70; C12Q 1/6844

(52) U.S. Cl. CPC C12Q 1/701; C12Q 1/6844

(57) ABSTRACT

A cross primer isothermal amplification primer set for detecting tobacco ringspot virus, a kit and application thereof are provided. The kit and the method provided by the disclosure have good specificity and negative reaction to quarantine viruses such as southern bean mosaic virus, tomato ringspot virus, Arabidopsis mosaic virus, bean pod mottle virus and the like. The real-time fluorescence detection method has high sensitivity and can detect the RNA template of the tobacco ringspot virus with the minimum concentration of 5 ng/μL. The lowest detectable concentration of RNA template was 0.5 ng/μL. It is very suitable for on-site detection of medical and health, food safety and import and export quarantine, and is easy to be popularized and applied in a wide range.

### REFERENCE TO SEQUENCE LISTING

The substitute sequence listing is submitted as an ASCII formatted text filed via Patent Center, with a file name of “Substitute_Sequence_Listing_GLP-US-SJDL169”, a creation date of Nov. 22, 2023, and a size of 8192 bytes. The substitute sequence Listing filed via Patent Center is a part of the specification and is incorporated in its entirety by reference herein.

### CROSS REFERENCE TO RELATED APPLICATION

This patent application claims the benefit and priority of Chinese Patent Application No. 202110557953.8 filed on May 21, 2021, the disclosure of which is incorporated by reference herein in its entirety as part of the present application.

### TECHNICAL FIELD

The invention belongs to the field of biotechnology, and more specifically, to a cross primer isothermal amplification primer set of soybean seed-borne quarantine virus tobacco ringspot virus, a rapid detection kit and an application thereof.

### BACKGROUND ART

Tobacco ringspot virus (TRSV) is a member of the genus Nepovirus of the subfamily Comovirinae. The virus particles are spherical with about 28 nm in diameter, and the nucleic acid is positive single-stranded RNA. The genome consists of two parts, RNA-Q and RNA-2. The virus is distributed in more than 50 countries and regions, including Japan, South Korea, the United States, Canada and Taiwan, China. It has a wide host range. According to De Zeeuw, it can infect 246 species of plants from 54 families, such as soybean and tobacco. TRSV infection can cause serious diseases, such as dwarfing soybean plants, making soybean pods undeveloped or abortive, causing ring spots and lines on tobacco leaves and dwarfing, and causing chlorotic spots on cucurbits and malformed fruits. TRSV can be transmitted by nematode disintegration, and it can be transmitted by seeds for a long distance. The seed transmission rate on soybean can reach 100%. TRSV has a high quarantine risk and is a second-class entry quarantine pest in China. In 2017, it was included in the list of plant entry quarantine pests in China.

Currently, the detection methods of tobacco ringspot virus mainly include ELISA, RT-PCR and real-time fluorescent TaqMan-MGB probe RT-PCR. However, these methods have limitations, such as the low detection limit of ELISA method, which is difficult to detect a small number of viruses in asymptomatic plants, and the ELISA method often fails to identify the types of viruses and has poor specificity. However, the detection method based on PCR technology has some defects, such as long reaction time, expensive thermal cycling instruments and professional operators, which are difficult to meet the needs of rapid detection of port quarantine.

Cross-priming isothermal amplification (CPA) is a nucleic acid amplification technology developed by Hangzhou Yousida Biotechnology Co., Ltd. and the first nucleic acid amplification technology with independent intellectual property rights in China. The CPA can be divided into two types: single crossing primer isothermal amplification (single crossing CPA) and double crossing primer isothermal amplification (double crossing CPA). Both type of reactions require a cross prim (CPF), two amplification primers (DR, MBR), two unstranded peripheral primers (BF, BR) and a DNA polymerase Bst with strand displacement activity. The double cross CPA has two cross primers, one more than the single cross CPA. The single cross CPA was produced after optimizing the double cross CPA mechanism. It can amplify four copies of genomic DNA in less than one hour, has high specificity, and is also a widely used cross-primer isothermal amplification technology. Since the production of CPA, it has been mainly used in the detection of Mycobacterium tuberculosis for the diagnosis of pulmonary tuberculosis. At present, the Yousida has developed a Mycobacterium tuberculosis nucleic acid detection kit (TB-CPA). It has high sensitivity and specificity, and can complete the detection and report within 2 hours, which is suitable for primary hospitals and health centers. In addition, the CPA has also carried out research in the field of food safety, such as the detection of genetically modified crops and the detection of foodborne pathogens Listeria monocytogenes. The invention establishes an African swine fever detection method combined with a test strip in the field of animal quarantine, detection methods of Prunus necrotic ringspot virus (PNRSV) and Cucumber green mottle mosaic virus (GGMMV) were established in the field of plant quarantine.

### SUMMARY

The invention provides a cross primer isothermal amplification rapid detection kit for tobacco ringspot virus and a primer aiming at the problems existing in the detection of the tobacco ringspot virus. The kit has the characteristics of high specificity, high sensitivity, simple operation, rapid detection and intuitive detection result.

The invention firstly discloses a cross primer isothermal amplification primer set for detecting tobacco ringspot virus. The cross primer isothermal amplification primer set includes a peripheral replacement primer TRBF4, a peripheral replacement primer TRBR4, an amplification primer TRDR4, an amplification primer TRMBR4 and a cross primer TRCPF4;

The nucleotide sequence of the peripheral replacement primer TRBF4 is shown in SEQ ID NO.1;

The nucleotide sequence of the peripheral replacement primer TRBR4 is shown in the SEQ ID NO.2;

The nucleotide sequence of the amplification primer TRDR4 is shown in the SEQ ID NO.3;

The nucleotide sequence of the amplification primer TRMBR4 is shown in SEQ ID NO.4;

The nucleotide sequence of the cross primer TRCPF4 is shown in SEQ ID NO.5.

As a preferred embodiment of the disclosure, the 5′ end of the amplification primer TRDR4 is labeled with 6-carboxyl fluorescein (FAM), and the 5′ end of the amplification primer TRMBR4 is labeled with biotin fluorophore (Biotin).

More preferably, the molar ratio of the peripheral displacement primer TRBF4, the peripheral displacement primer TRBR4, the amplification primer TRDR4, the amplification primer TRMBR4 and the cross primer TRCPF4 is 1:1:3:3:5.

A cross-primer isothermal amplification reagent for detecting the tobacco ringspot virus is also provided, which includes the cross-primer isothermal amplification primer set.

The final concentrations of the peripheral displacement primer TRBF4 and the peripheral displacement primer TRBR4 in the amplification reagent are both 0.2 μmol/L.

The final concentrations of the amplification primer TRDR4 and the amplification primer TRMBR4 in the amplification reagent are both 0.6 μmol/L;

The final concentration of the cross primer TRCPF4 in the amplification reagent is 1 μmol/L.

The invention also provides a cross primer isothermal amplification kit for detecting the bean pod mottle virus, which includes the primer set or the amplification reagent.

An application of the primer set or the amplification reagent or the kit in detecting whether a sample to be detected is infected with the tobacco ringspot virus are also provided.

The invention further provides a method for detecting whether a sample to be detected is infected with tobacco ringspot virus, which comprises the following steps:
performing a cross primer isothermal amplification on a sample to be detected by adopting the primer set or the amplification reagent or the kit;
monitoring in real time, by the fluorescent dye and a real-time fluorescence instrument, and observing a fluorescence curve;
if the amplification curve does not appear, the sample to be detected is not infected by the tobacco ringspot virus; and
if the amplification curve appears, the sample to be detected is infected by the tobacco ringspot virus.

A method for detecting whether a sample to be detected is infected with tobacco ringspot virus is further provided, which includes the following steps:
performing a cross primer isothermal amplification on a sample to be detected by adopting the primer set or the amplification reagent or the kit,
detecting the amplification product by using a nucleic acid test strip, and observing the test strip after 2 to 5 minutes;

If a blue strip appears on the quality control line and no strip appears on the detection line, the sample to be detected is not infected by the tobacco ringspot virus; and
if a blue or red strip appears on the quality control line and a red strip appears on the detection line, the sample to be detected is infected by tobacco ringspot virus.

Preferably, the cross primer isothermal amplification reaction condition is 60° C., and the reaction time is 90 minutes.

Preferably, the template of the cross primer isothermal amplification is RNA.

Compare with that prior art, beneficial effects are:
(1) because that cost of the cross-primer isothermal amplification detection method is low, and the isothermal amplification is realize at 60° C. by using AMV reverse transcriptase and Bst DNA polymerase (the amplification of an RNA template can be directly realize without a separate reverse transcription step), the kit provided by the invention is convenient to use and has low use cost.
(2) the reaction result obtained by the kit provided by the invention is intuitive and accurate without complicated operation. The real-time fluorescence detection method only needs to add SYTO 16 green nucleic acid dye in the reaction system of the kit, the real-time fluorescence instrument is used to monitor the amplification process in real time, and the result can be judged by the amplification curve. The nucleic acid test strip detection method only needs to label 6-carboxyl fluorescein set on that amplification primer TRDR, label biotin on the other amplification primer TRMBR, and then uses the disposable nucleic acid test strip to detect the reaction product, thereby being capable of accurate, intuitive and rapid interpretation.
(3) the kit provided by the disclosure has good specificity and has negative reaction to southern bean mosaic virus, tomato ringspot virus, Arabidopsis mosaic virus, bean pod mottle virus and the like. The real-time fluorescence detection method can detect the total RNA template of the tobacco ringspot virus at a minimum concentration of 5 ng/μL, and the nucleic acid test strip detection method can detect the total RNA template of the tobacco ringspot virus at a minimum concentration of 0.5 ng/μL Tobacco ringspot virus total RNA template.
(4) the tobacco ringspot virus cross primer isothermal amplification kit can rapidly and sensitively detect the tobacco ringspot virus. The kit has the advantages of simple operation, low cost, easy observation of reaction results and good specificity, is very suitable for field detection of medical health, food safety and import and export quarantine, and is easy to popularize and apply in a wide range.

### BRIEF DESCRIPTION OF THE DRAWINGS

FIG. 1 is a diagram of a result of screening and detecting a prim set of tobacco ringspot virus by isothermal amplification of real-time fluorescent cross primers of the disclosure. The real-time fluorescence was monitored every 60 s, and each cycle was 1 min. The S1 to S5 correspond to the amplification curve of real-time fluorescent cross-primer isothermal amplification of TRSV primer set S1 to primer set S5 in turn, and Negative control (NC) is the amplification curve with water as the template.

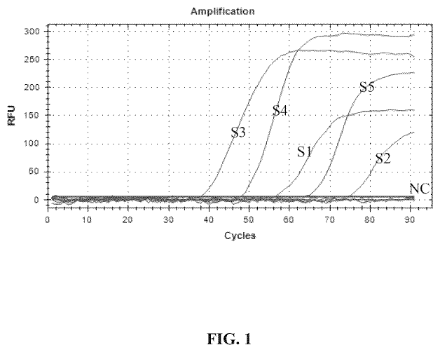

FIG. 2 is a graph showing the result of evaluating the specificity of the S4 prim set for detecting TRSV by using a real-time fluorescent cross-primer isothermal amplification method. The amplification curve of total RNA templates and wat templates (NC) of southern bean mosaic virus, tomato ringspot virus, Arabidopsis mosaic virus, tobacco ringspot virus and bean pod mottle virus detected by using S3 as a real-time fluorescent cross-primer isothermal amplification prim set.

FIG. 3 is a graph showing the results of a sensitivity test for detecting TRSV by the real-time fluorescent cross-primer isothermal amplification method provided by the disclosure. The total RNA template of the TRSV was continuously diluted in a 10-fold gradient at a concentration of 50 ng/μL, and seven concentration gradients were established, with 10⁻⁵-10⁻⁶ corresponding to the continuously diluted RNA template of TRSV in a 10-fold gradient in turn. The template concentrations were 50 ng/μL, 5 ng/μL, 0.5 ng/μL, 5×10⁻² ng/μL, 5×10⁻³ ng/μL, and 5×1⁻⁴ ng/μL respectively. The NC is a negative control using water as a template.

FIG. 4 is a graph showing the result of a TRSV specificity test using the cross-primer isothermal amplification kit provided by the disclosure in combination with a nucleic acid test strip method. Wherein the numbers from 1 to 5 sequentially correspond to the detection results of southern bean mosaic virus, tomato ringspot virus, Arabidopsis mosaic virus, tobacco ringspot virus and bean pod mottle virus total RNA templates, and the NC is the detection result using water as a template.

FIG. 5 is a graph showing the result of a sensitivity test for detecting TRSV by using the cross-primer isothermal amplification kit provided by the disclosure in combination with a nucleic acid test strip method. The 10⁵-10⁶ sequentially corresponds to a tobacco ringspot virus RNA template which is continuously diluted by a 10-fold gradient. The template concentrations were 50 ng/μL, 5 ng/μL, 0.5 ng/μL, 5×10⁻² ng/μL, 5×10⁻³ ng/μL, and 5×1⁻⁴ ng/μL. The NC is a negative control using water as a template.

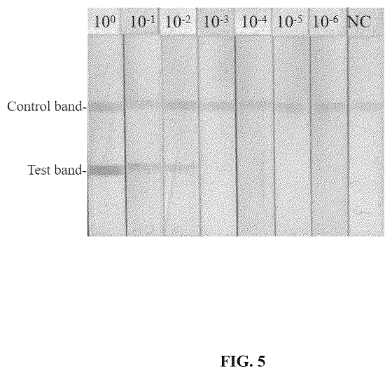

FIG. 6 is a graph showing the results of a sensitivity test for the detection of TRSV by Taq-Man real-time fluorescent RT-PCR. The 10⁻⁵-10⁻⁶ sequentially corresponds to a tobacco ringspot virus RNA template which is continuously diluted by a 10-fold gradient. The template concentrations were 50 ng/μL, 5 ng/μL, 0.5 ng/μL, 5×10⁻² ng/μL, 5×10⁻³ ng/μL, and 5×1⁻⁴ ng/μL. The NC is a negative control using water as a template.

### DETAILED DESCRIPTION OF THE EMBODIMENTS

Hereinafter, the embodiments of the disclosure will be described in detail in combination with the examples, so that the experimental process of how to apply the technical means to solve the technical problems and achieve the technical effects of the disclosure can be fully understood and implemented.

Materials involved in the embodiments:
a. The primer was synthesized by Shanghai Paisenuo Biological Co., Ltd. The Bst DNA polymerase was purchased from New England Corporation; AMV reverse transcriptase was purchased from Promega; Pfu DNA polymerase was purchased from Transgene Corporation; Betaine was purchased from Shanghai Sangon Bioengineering Co., Ltd. SYTO™ 16 Green Nucleic Acid Dye was purchased from Thermo Fisher; Tiosbio® disposable nucleic acid test strip (lateral flow dipstick, LFD) is Purchased from Beijing Baoying Tonghui Biotechnology Co., Ltd. (Item No.: JY0201); Plant total RNA extraction kit was purchased from TaKaRa company (TaKaAa minibest universal RNA extraction kit, 9767); The cDNA synthesis kit was also purchased from TaKaRa (Prime Script™ II 1st strand cDNA synthesis kit, 6210A).
b. Tobacco ringspot virus (TRSV) was used as the test sample, and Southern bean mosaic virus (SBMV), Tomato ringspot virus (ToRSV), Arabis mosaic virus (ArMV) and Bean pod mottle virus (BPMV) were used as the control samples in the specificity evaluation, which are also a burley tobacco leaf with virus. These experimental samples were provided by the Chinese Academy of Inspection and Quarantine.

### Example 1 Design and Synthesis of Cross-Primed Isothermal Amplification Primer

### I: Sequence Acquisition

### 1. Extraction of RNA

50 mg of tobacco leaves infected with SBMV, ToRSV, ArMV, TRSV and BPMV were used to extract the total RNA of TRSV virus samples and negative control according to the operation instructions of plant total RNA extraction kit.

2. Synthesis of cDNA

3 μL of total RNA of each of the five viruses extracted above was used to reverse transcribe the cDNAs of the five viruses using a two-step kit (Prime Script™ II 1st strand cDNA synthesis kit, 6210A).

### 3. Design and Synthesis of CP Gene Prim

The genome sequence of tobacco ringspot virus (GenBank accession number: AY363727) was downloaded from GenBank, and the primers of tobacco ringspot virus CP gene were designed by using software Gencious 11.1.5 based on the primer design principles of GC content 40%-50%, TM value 50%-60% and avoiding repetitive sequences. The nucleotide sequence of the upstream amplification primer TRF is shown in SEQ ID NO.26, and the nucleotide sequence of the downstream amplification primer TRR is shown in SEQ ID NO.27 (TRF-AACCTATGAAGAGGGAAATGC, TRR-CCAATCCTGGTTTGAAGAATC). The primers were synthesized by Shanghai Sunny Biotechnology Co., Ltd.

### 4. CP Amplification

TRSV-CP gene was amplified by PCR using the screened primers and Pfu DNA polymerase (2× TransStart FastPfu PCR Super Mix, full gold). The 50 μL reaction system was as follows: 2× Super Mix 25 μL, template cDNA 1 μL, TRF and TRR 1 μL each, primer final concentration 0.5 μmol/L, ddH₂O is supplemented to 50 μL. The PCR amplification conditions were as follows: pre-deformation at 94° C. for 3 min; denaturation at 98° C. for 10 s, annealing at 53° C. for 30 s, extension at 72° C. for 120 s, 34 cycles: and extension at 72° C. for 8 min.

### 5. Sequence Acquisition (Ligation of Transformed Clones)

After the PCR product was verified by agarose gel electrophoresis, the SanPrep Column DNA Gel Extraction Kit (Sangon) was used to extract the DNA, and the purified DNA was connected to the A-tail to prepare a 10 μL reaction system, which includes 0.1 μL of Ex Taq enzyme, 1 μL of 10×PCR buffer, 0.8 μL of dNTP, 8.1 μL purified DNA, and then ligated to PMD19T vector (TakaRa) to configure 10 μL reaction system, including 5 μL Solution I. 0.5 μL PMD19T vector, 4.5 μL cDNA, prepared on ice, ligated at 16° C. for 12 H. The ligation product was then transformed into competent cells (T-Fast Competent E. Coli). 200 μL of transformed bacterial solution was taken and 40 μL X-gal (20 mg/ml) and 16 μL IPTG (50 mg/ml) were added and mixed well. Then they are spread on ampicillin (Amp) resistant solid medium, and were placed in 37° C. incubator for 10-12 H. The white single colony were picked and shaked.

The amplified bacterial solution was sent for sequencing by Shanghai Sangon Bioengineering Co., Ltd. to obtain the TRSV-CP gene sequence, which was determined as the TRSV-CP gene sequence after NCBI sequence comparison.

### Design and Synthesis of Primer

### Design of Pecific Primer

The software Geneious Primers 11.1.5 is used, based on the principle of CPA primer design (the cross primer is composed of the amplification primer TRMBR at the 5′ end and the sequence of about 20-22 BP complementary to the target gene at the 3′ end), the total length of the cross primer is about 40 BP. The other four primers (peripheral replacement primer TRBF/TRBR and amplification primer TRDR/TRMBR) were all about 18-20 BP, and the CG content of each primer should be controlled between 40% and 60% to ensure that the Tm values of the primers would not be too different. When designing the primers, we should try our best to avoid the influence of dimers between primers on the amplification results, and at the same time try our best to avoid a large number of repetitive sequences and mutation sites), and designed five sets of CPA primers for TRSV-CP gene sequence (Table 1), each set of primers contains five primers. It contains a cross primer TRCPF (40 BP), a single-stranded primer TRBF/TRBR (19 BP) and an amplification primer TRDR/TRMBR (19 BP). The nucleotide sequence of the S4 set peripheral replacement primer TRBF4 is shown as a SEQ ID NO.1, the nucleotide sequence of the peripheral replacement primer TRBR4 is shown as a SEQ ID NO.2, the nucleotide sequence of an amplification primer TRDR4 is shown as an SEQ ID NO.3, the nucleotide sequence of the amplification primer TRMBR4 is shown as SEQ ID NO.4, the nucleotide sequence of the cross primer TRCPF4 is shown as SEQ ID NO.5, and the nucleotide sequence corresponding to the S1 set is shown as SEQ ID NO.6 to SEQ ID NO.10; The nucleotide sequence corresponding to the S2 set is shown as SEQ ID NO.11-SEQ ID NO.15. The nucleotide sequence corresponding to the S3 set is shown as SEQ ID NO.16 to SEQ ID NO.20. The nucleotide sequence corresponding to the S5 set is shown in the SEQ ID NO.20 to the SEQ ID NO.25.

The primer synthesis was completed by Shanghai Sunny Biotechnology Co., Ltd.

### Example 2 Application of Cross-Primed Isothermal Amplification Primers for Detection of Tobacco Ringspot Virus

### 1. Primer Screening

The total RNA of tobacco ringspot virus (TRSV) was used as a template to screen and detect five primer sets of TRSV by real-time fluorescent cross-primer isothermal amplification. The results are shown in Figure (1): the primer set S4 (Table 1) of the cross-primer isothermal amplification for detecting tobacco ringspot virus was determined by real-time fluorescent cross-primer isothermal amplification detection. The primer set S4 includes peripheral displacement primers (TRBF4 and TRBR4), two amplification primers (TRDR4 and TRMBR4), and one cross primer S4 (TRCPF4).

TABLE 1
Primer information of cross primer isothermal amplification
reaction of tobacco ringspot virus

| Primer set | Name | Sequence (5′-3′) |
|---|---|---|
| S1 | TRBF1 | CTCTGCCAGCACTGCTCAT (SEQ ID NO. 6) |
| | TRBR1 | CTTGGGAATGCAAACCACC (SEQ ID NO. 7) |
| | TRDR1 | GTCCCACAACAAGTGGGAT (SEQ ID NO. 8) |
| | TRMBR1 | GCTTTCCTTTCTTCGCATC (SEQ ID NO. 9) |
| | TRCPF1 | GCTTTCCTTTCTTCGCATCGTATGTGTGCTGTG |
| | | ACGGTTG (SEQ ID NO. 10) |
| S2 | TRBF2 | GGTGTGCAAAGGGCATTGT (SEQ ID NO. 11) |
| | TRBR2 | CTTGGTAGGCAGTTCAAAC (SEQ ID NO. 12) |
| | TRDR2 | AAACCTGCAAAGGCGTTGC (SEQ ID NO. 13) |
| | TRMBR2 | ATCATCAAAGGTGCACGCT (SEQ ID NO. 14) |
| | TRCPF2 | ATCATCAAAGGTGCACGCTCATCCCACTTTTA |
| | | CAGTGAGG (SEQ ID NO. 15) |
| S3 | TRBF3 | GCAGATGTGCATGAATGGC (SEQ ID NO. 16) |
| | TRBR3 | CCATGTCCACATGCATAGT (SEQ ID NO. 17) |
| | TRDR3 | TGGGTAACTACATTTGCCC (SEQ ID NO. 18) |
| | TRMBR3 | CTTGATTTGTGGACGCAAC (SEQ ID NO. 19) |
| | TRCPF3 | CTTGATTTGTGGACGCAACTGCTATGGGGAA |
| | | CTTACAGGA (SEQ ID NO. 20) |
| S4 | TRBF4 | GATCCAACTATGACGTGGC (SEQ ID NO: 1) |
| | TRBR4 | GCCCGGGAATATGAAATGG (SEQ ID NO. 2) |
| | TRDR4 | AGGTTGACCCGTCAAGTTT (SEQ ID NO. 3) |
| | TRMBR4 | TGACGGGACCTCCAAAATT (SEQ ID NO. 4) |
| | TRCPF4 | TGACGGGACCTCCAAAATTCTAATTGGGTGA |
| | | CACTTTCGC (SEQ ID NO. 5) |
| S5 | TRBF5 | TCTAGGAATCTTGGGTGGT (SEQ ID NO. 21) |
| | TRBR5 | TCAAATTGGCCATCTCCGT (SEQ ID NO. 22) |
| | TRDR5 | AGGTGCTTCGTGTTCCCAT (SEQ ID NO. 23) |
| | TRMBR5 | CATAACACCTGACGTAAGG (SEQ ID NO. 24) |
| | TRCPF5 | CATAACACCTGACGTAAGGGTCACTATAAGC |
| | | GGAAGTGTC (SEQ ID NO. 25) |

### 2. Establishment of Real-Time Fluorescent Cross-Primer Isothermal Detection Method

(1) Real-time fluorescence RT-CPA Real-time fluorescence instrument: CFX Connect Real-Time System (Bio-Rad, America) was used.
(2) isothermal amplification reaction system of tobacco ringspot virus cross prim

TABLE 2
Isothermal Amplification Reaction System
of Tobacco Ringspot Virus Cross Primer

| Ingredient | Volume (μL) | Final concentration | |
|---|---|---|---|
| 10× ThermoPol buffer (20 | 0.6 | 0.6 | mM |
| mmol/L) | | | |
| dNTP (2.5 mmol/L) | 0.8 | 0.1 | mM |
| Betaine (5 mol/L) | 0.5 | 125 | mM |
| MgSO4 (100 mmol/L) | 0.4 | 2 | mM |
| TRCPF (20 μM) | 1 | 1 | μM |
| TRDR (20 μM) | 0.6 | 0.6 | μM |
| TRMBR (20 μM) | 0.6 | 0.6 | μM |
| TRBF (20 μM) | 0.2 | 0.2 | μM |
| TRBR (20 μM) | 0.2 | 0.2 | μM |
| AMV (10 U/μL) | 1 | 10 | U |
| Bst DNA Polymerase (8 U/μL) | 1 | 8 | U |
| RNA (50 ng/μL) | 1 | 50 | ng |
| ddH₂O | Add to 20 | | |

The real-time fluorescence reaction system requires adding 0.5 μL of SYTO™16 green nucleic acid dye (100 μmol/L, ThermoFisher) to the reaction system in Table 2, and the rest is consistent with the reaction system in Table 2.

### (3) Result Judgment:

Whether an amplification curve is present,
Negative—no amplification curve appears and no fluorescence signal is collected;
Positive—the amplification curve appears and the fluorescence signal is collected;
Invalid—a condition in which the amplification curve is messy and broken.

### (4) Reaction Procedure of the Real-Time Fluorescence Method:

The real-time fluorescence cross-primer isothermal amplification was carried out at 60° C. for 90 min, and the fluorescence was monitored every 60 s, each 1 min as a cycle, and the FAM channel (excitation and emission wavelengths at 450-490 nm) in the first channel was selected as the fluorescence absorption channel.

### III. Establishment of Test Strip Detection Method

Cross-primed isothermal amplification in combination with nucleic acid test strips requires the use of a PCR instrument (Bio-Rad T100™ Thermal Cycler, America). The reaction system of the detection method of the cross primer isothermal amplification combined with nucleic acid test strip is the same as that shown in Table 2, and the amplification primers TRDR and TRMBR need to be labeled in the detection method system of the cross primer isothermal amplification combined with nucleic acid test strip. The 5′ end of the amplification primer TRDR was labeled with 6-carboxyfluorescein (FAM), and the 5′ end of the other amplification primer TRMBR was labeled with Biotin.

The detection method of the cross primer isothermal amplification combined with the nucleic acid test strip comprises the following steps of: reacting for 90 minutes at a constant temperature of 60′C, inserting the nucleic acid test strip into a PCR tube after the reaction is finished, and observing a result after 2 to 5 minutes.

The result identification method of the nucleic acid test strip is as follows:

Negative (−): Only one blue band appears in the control band, and no band appears in the Test band. It was proved that the samples tested were not infected by tobacco ringspot virus.

Positive (+): two bands appear, one red or blue band appears on the quality control line, and one red band appears on the detection line. It was proved that the samples were infected by tobacco ringspot virus.

Analysis of specificity and sensitivity of cross prim isothermal amplification detection system

### 1. Real-Time Fluorescence Detection Method

### (1) Specificity Analysis

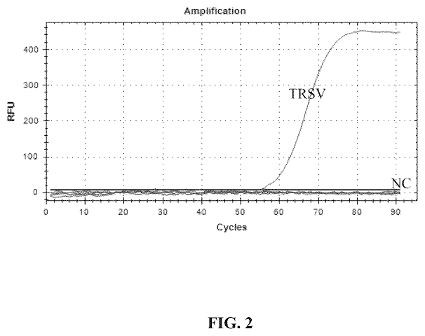

The specificity of the system was detected by using the total RNA of five soybean seed borne quarantine viruses (Southern Bean Mosaic Virus, Tomato Ringspot Virus, Arabidopsis Mosaic Virus, Tobacco Ringspot Virus and Bean Pod Mottle Virus) as the template, and water as the negative control. By using a cross-primer isothermal real-time fluorescence detection system, the specificity of primer set S4 for detecting tobacco ringspot virus was evaluated, and the amplification reaction process was monitored in real time by real-time fluorescent PCR instrument. The specificity evaluation result of the primer set S4 is shown in FIG. 2: the cross primer isothermal amplification reaction product with the total RNA of the tobacco ringspot virus as a template shows an amplification curve; No fluorescent amplification curves were found in the cross-primer isothermal amplification reactions using southern bean mosaic virus, tomato ringspot virus, Arabidopsis mosaic virus and bean pod mottle virus total RNA as templates, and no amplification curves were found in the negative control reactions using water as templates. The results showed that the primer set S4 detection system had good specificity and could specifically detect tobacco ringspot virus. And finally, the prim set S4 is determined to be the prim set in the cross primer isothermal amplification kit for detecting the tobacco ringspot virus.

### (2) Sensitivity Analysis

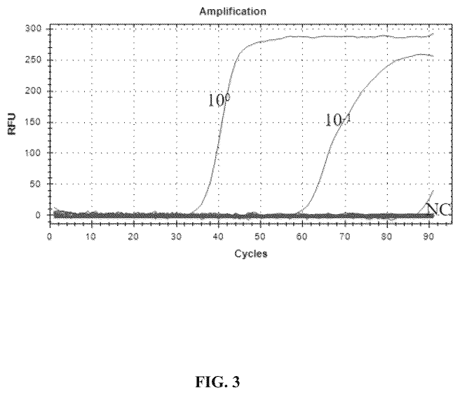

RNa se-Free ddH₂O was used to continuously dilute the total RNA of TRSV sample (50 ng/μL) by 10-fold gradient, and 7 gradients were set up. The total RNA of TRSV diluted according to the gradient was used as the template, and water was set as the negative control. The results are shown in FIG. 3, wherein amplification curves appeared in 10⁰ and 10⁻¹ dilution gradients of TRSV total RNA templates, that is, TRSV total RNA templates with template concentrations of 50 ng/μL and 5 ng/μL could be amplified. The detection limit of real-time fluorescence CPA was 5 ng/μL.

### 2. Nucleic Acid Test Strip Detection Method

### (1) Specificity Analysis

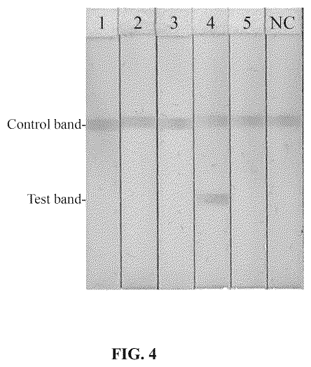

The specificity of the system was also detected by using the total RNA of five soybean seed borne quarantine viruses (Southern Bean Mosaic Virus, Tomato Ringspot Virus, Arabidopsis Mosaic Virus, Tobacco Ringspot Virus and Bean Pod Mottle Virus) as templates, and water as a negative control. The reaction system was shown in Table 2, and the products were detected using disposable nucleic acid test strips. The results are shown in FIG. 4: the cross primer isothermal amplification reaction product with the total RNA of tobacco ringspot virus as the template showed two bands, and both the quality control line and the detection line showed red bands. The total RNA of southern bean mosaic virus, tomato ringspot virus, Arabidopsis mosaic virus and bean pod mottle virus were used as templates for cross primer isothermal amplification. Only one blue band appears on the quality control line, and no band appears on the detection line. The results showed that the nucleic acid test strip cross-primer isothermal amplification detection system had good specificity and could specifically detect tobacco ringspot virus.

### (2) Sensitivity Analysis

RNa se-Free ddH₂O was used to continuously dilute the total RNA of TRSV sample (50 ng/μL) by 10-fold gradient, and 7 gradients were set up. The total RNA of TRSV diluted according to the gradient was used as the template, and water was set as the negative control. The disposable nucleic acid test strip was used to detect the product. The results obtained are shown in Figure (5): 10⁰, 10⁻¹ and 10⁻² dilution gradients, i.e. concentrations of 50 ng/μL, 5 ng/μL and 0. The cross-primer isothermal amplification of 5 ng/μL of TRSV total RNA template produced two bands, and the sensitivity of the CPA-nucleic acid test strip for TRSV detection was 0.5 ng/μL.

### 3. Taq-Man Real-Time Fluorescent RT-PCR

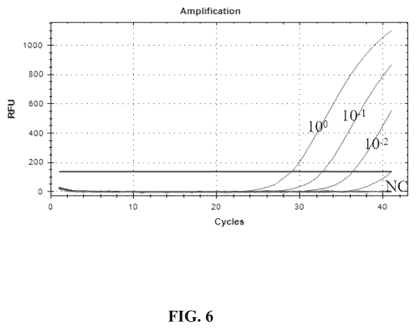

In order to compare the sensitivity of the cross-primer isothermal amplification detection method established in this study with that of TaqMan-MGB real-time fluorescent RT-PCR, TRSV primers and probes were designed according to national standards (Table 3, SEQ ID NO.28 to SEQ ID NO.30). The serial 10-fold dilutions of TRSV total RNA were used for RT-qPCR reaction, and the sensitivity differences between cross-primer isothermal amplification and RT-qPCR were analyzed and compared. The RT-qPCR 50 μL reaction system is shown in (Table 4). The reaction procedure of RT-qPCR was: reverse transcription at 50° C. for 30 min; Predenaturation at 95° C. for 3 min; Predeformation at 95° C. for 15 s, extension at 60° C. for 30 s, 40 cycles (fluorescence was collected at the cycle step). The detection results of Taq-Man real-time fluorescent RT-PCR are shown in FIG. 6. Amplification curves appeared in three dilution gradients of 10⁰-10⁻². The sensitivity of Taq-Man real-time fluorescent RT-PCR for TRSV detection can reach 5 ng/μL.

TABLE 3
primer and probes for RT-qPCR detection of TRSV

| Primer and probe | Sequence (5′-3′) |
|---|---|
| Primer TRSV-5P | TCCATGTTGTCCATATCTTA (SEQ ID NO. 28) |
| Primer TRSV-3P | AGAAGAAACACTCTTGACACT (SEQ ID NO. 29) |
| Probe | FAM-CCGCTTATAGTGCCAGACCA-TAMRA (SEQ ID |
| | NO. 30) |

TABLE 4
RT-qPCR reaction system

| Reagent | Volume | |
|---|---|---|
| 2× FastKing One Step Probe RT-qPCR MasterMix | 25 | μL |
| 25× FastKing Enzyme Mix | 2 | μL |
| TRSV-5P (10 μM) | 1.25 | μL |
| Primer TRSV-3P (10 μM) | 1.25 | μL |
| TRSV Probe (10 μM) | 1.25 | μL |
| RNA template (50 ng/μL) | 1 | μL |

| | |
|---|---|
| RNase-Freedd H₂O | Complement |

The above embodiments only represent several embodiments of the present invention, and the description thereof is more specific and detailed, but should not be construed as limiting the scope of the present invention. It should be noted that it will be apparent to those of ordinary skill in the art that numerous variations and modifications may be made without departing from the spirit of the invention. All of these fall within the scope of the present invention. Therefore, the scope of protection of this invention patent should be determined by the following claims.

What is claimed is:

1. A cross primer isothermal amplification primer set for detecting tobacco ringspot virus, characterized in that the cross primer isothermal amplification primer set comprises a peripheral replacement primer TRBF4, a peripheral replacement primer TRBR4, an amplification primer TRDR4, an amplification primer TRMBR4 and a cross primer TRCPF4:
a nucleotide sequence of the peripheral replacement primer TRBF4 is shown in SEQ ID NO.1;
the nucleotide sequence of the peripheral replacement primer TRBR4 is shown in the SEQ ID NO.2;
the nucleotide sequence of the amplification primer TRDR4 is shown in the SEQ ID NO.3;
the nucleotide sequence of the amplification primer TRMBR4 is shown in SEQ ID NO.4; and
the nucleotide sequence of the cross primer TRCPF4 is shown in SEQ ID NO.5.

2. The cross-primer isothermal amplification primer set for detecting tobacco ringspot virus of claim 1, wherein a 5′ end of the amplification primer TRDR4 is labeled with 6-carboxyl fluorescein, and the 5′ end of the amplification primer TRMBR4 is labeled with biotin fluorophore.

3. The cross primer isothermal amplification primer set for detecting tobacco ringspot virus of claim 1, wherein a molar ratio of the peripheral replacement primer TRBF4, the peripheral replacement primer TRBR4, the amplification primer TRDR4, the amplification primer TRMBR4 and the cross primer TRCPF4 is 1:1:3:3:5.

4. A cross-primer isothermal amplification reagent for detecting tobacco ringspot virus, comprising the cross-primer isothermal amplification primer set according to claim 1;
final concentrations of the peripheral displacement primer TRBF4 and the peripheral displacement primer TRBR4 in the amplification reagent are both 0.2 μmol/L;
final concentrations of the amplification primer TRDR4 and the amplification primer TRMBR4 in the amplification reagent are both 0.6 μmol/L; and
a final concentration of the cross primer TRCPF4 in the amplification reagent is 1 μmol/L.

5. A cross-primer isothermal amplification kit for detecting bean pod mottle virus, comprising the primer set of claim 1.

6. An application of the primer set of claim 1 in detecting whether a sample to be detected is infected with tobacco ringspot virus.

7. A method for detecting whether a sample to be detected is infected with tobacco ringspot virus, comprising:
performing cross-primer isothermal amplification on a sample to be detected by using the primer set of claim 1;
monitoring in a real time, by a fluorescent dye and a real-time fluorescence instrument, and observing a fluorescence curve;
if the amplification curve does not appear, the sample to be detected is not infected by the tobacco ringspot virus; and
if the amplification curve appears, the sample to be detected is infected by the tobacco ringspot virus.

8. A method for detecting whether a sample to be detected is infected with tobacco ringspot virus, comprising:
performing cross-primer isothermal amplification on a sample to be detected by the primer set of claim 1;
an amplification product of the kit is detected by a fluorescently labeled nucleic acid test strip, and the test strip is observed after 2 to 5 minute;
if a blue strip appears on the quality control line and no strip appears within the detection limit, the sample to be detected is not infected by the tobacco ringspot virus; and
if a blue or red strip appears on the quality control line and a red strip appears on the detection line, the sample to be detected is infected by tobacco ringspot virus.

9. The method of claim 7, wherein the cross-primer isothermal amplification reaction is carried out at 60° C. for 90 min.

10. The method of claim 7, wherein the template of the cross-primer isothermal amplification is RNA.
